(19) United States
(12) Patent Application Publication
(10) Pub. No.: US 2026/0346016 A1
(43) Pub. Date: Nov. 5, 2026

(54) IMPROVED FEEDBACK CONTROL OF NEURAL STIMULATION

(71) Applicant: Saluda Medical Pty Limited, Macquarie Park, New South Wales (AU)

(72) Inventors: Peter Scott Vallack SINGLE, Macquarie Park (AU); Zubin Zarir NANAVATI, Macquarie Park (AU); Julian Frank Andrew MAGAREY, Macquarie Park (AU)

(21) Appl. No.: 19/488,135

(22) PCT Filed: May 30, 2024
(86) PCT No.: PCT/AU2024/050562

§ 371 Date: Nov. 26, 2025

(30) Foreign Application Priority Data
May 30, 2023 (AU) 2023901688

(51) Int. Cl. A61N 1/36; A61N 1/05

(52) U.S. Cl. CPC A61N 1/36139; A61N 1/0551; A61N 1/36062; A61N 1/36067; A61N 1/36075; A61N 1/3615

(57) ABSTRACT

Disclosed is an implantable neuromodulation device for controllably delivering neural stimuli. The device having a stimulus source (100) to provide the neural stimuli to a neural pathway of a patient (108) in order to evoke a neural response. The control unit of the devices estimates one or more defining parameters of an intermediate activation plot corresponding to a current posture of the patient from at least a plurality of key parameters of a respective plurality of reference activation plots corresponding to respective reference postures. Or the control unit estimates defining parameters of reference activation plots. Based on these parameters, the device uses a feedback controller to then maintain a recruitment value of the provided neural stimuli at or near a recruitment target value.

### INCORPORATION BY REFERENCE

The present application claims priority from Australian provisional patent application 2023901688 filed on 30 May 2023, the contents of which are incorporated herein by reference in their entirety.

### TECHNICAL FIELD

The present invention relates to closed-loop neural stimulation and in particular to methods and devices for feedback control of neural stimulation that maintain constant neural activation in the target neural tissue.

### BACKGROUND OF THE INVENTION

There are a range of situations in which it is desirable to apply neural stimuli in order to alter neural function, a process known as neuromodulation. For example, neuromodulation is used to treat a variety of disorders including chronic neuropathic pain, Parkinson's disease, and migraine. A neuromodulation device applies an electrical pulse (stimulus) to neural tissue (fibres, or neurons) in order to generate a therapeutic effect. In general, the electrical stimulus generated by a neuromodulation device evokes a neural response known as an action potential in a neural fibre which then has either an inhibitory or excitatory effect. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a device typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in an orthodromic direction (in afferent fibres this means towards the head, or rostral) and in an antidromic direction (in afferent fibres this means towards the cauda, or caudal) directions. Action potentials propagating along Aβ (A-beta) fibres being stimulated in this way may inhibit the transmission of pain from a region of the body innervated by the target neural fibres (the dermatome) to the brain. To sustain the pain relief effects, stimuli are applied repeatedly, for example at a frequency in the range of 30 Hz-100 Hz.

For effective and comfortable neuromodulation, it is necessary to maintain stimulus intensity above a recruitment threshold. Stimuli at intensities below the recruitment threshold will fail to recruit (or activate) sufficient neurons to generate action potentials with a therapeutic effect. In some neuromodulation applications, response from a single class of fibre is desired, but the stimulus waveforms employed can evoke action potentials in other classes of fibres which cause unwanted side effects. In pain relief, it is therefore desirable to apply stimuli with intensity below a discomfort threshold, above which uncomfortable or painful percepts arise due to over-recruitment of Aβ fibres or recruitment of undesired fibre classes. When recruitment is too large, Aβ fibres produce uncomfortable sensations. Stimulation at high intensity may even recruit Aδ (A-delta) fibres, which are sensory nerve fibres associated with acute pain, cold and heat sensation. It is therefore desirable to maintain stimulus intensity within a therapeutic range between the recruitment threshold and the discomfort threshold.

The task of maintaining appropriate neural recruitment is made more difficult by electrode migration (change in position over time) or postural changes of the implant recipient (patient), either of which can significantly alter the neural recruitment arising from a given stimulus, and therefore the therapeutic range. There is room in the epidural space for the electrode array to move, and such array movement from migration or posture change alters the electrode-to-cord distance and thus the recruitment efficacy of a given stimulus. Moreover, the spinal cord itself can move within the cerebrospinal fluid (CSF) with respect to the dura. During postural changes, the amount of CSF or the distance between the spinal cord and the electrode can change significantly. This effect is so large that postural changes alone can cause a previously comfortable and effective stimulus regime to become either ineffectual or painful.

Attempts have been made to address such problems by way of feedback or closed-loop control, such as using the methods set forth in International Patent Publication No. WO2012/155188 by the present applicant. Feedback control seeks to compensate for relative nerve/electrode movement by controlling the intensity of the delivered stimuli so as to maintain neural recruitment at or near a target value. The intensity of a neural response evoked by a stimulus may be used as a feedback variable representative of the amount of neural recruitment. A signal representative of the neural response may be sensed by a measurement electrode in electrical communication with the recruited neural fibres, and processed to obtain the feedback variable. Based on the response intensity, the intensity of the applied stimulus may be adjusted to bring the response intensity closer to a target value.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be measured at a measurement electrode as a positive peak potential, then a negative peak, followed by a second positive peak. This morphology is caused by the region of activation passing the measurement electrode as the action potentials propagate along the individual fibres. Approaches proposed for obtaining a neural response measurement are described by the present applicant in International Patent Publication No. WO2012/155183, the content of which is incorporated herein by reference.

However, a feedback control algorithm that simply seeks to maintain the measured response intensity at or near a target value representative of pain relief will not generally maintain constant neural recruitment (or activation) as posture changes. This is because a change in distance between the measurement electrode and the site of activation on the dorsal column, such as induced by posture change, affects the measured response intensity even if there is no change to the neural activation. In other words, an ECAP-controlled feedback loop maintains the ECAP amplitude at the measurement site, not at the activation site, which is where the neural activation actually needs to be maintained, and changes in posture cause these two sites to diverge. The result is that an ECAP-controlled feedback loop may adjust stimulus intensity in response to a posture-induced change in measured ECAP amplitude, even if there is no change to the neural activation.

One method of overcoming this difficulty would be to monitor the distance between the measurement electrode and the spinal cord, and normalise out the effect of any changes to this distance on the measured neural response intensity. However, such distance measurement would require at least one sensing modality in addition to the measurement of neural responses and would therefore add cost to a neuromodulation device. The extra processing required to accomplish the monitoring and the normalisation would also consume additional power and therefore shorten the battery life of the device.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood to mean that the element may be any one of the listed options, or may be any combination of two or more of the listed options.

### SUMMARY OF THE INVENTION

Disclosed herein are devices and methods for feedback control of neuromodulation that maintain constant neural activation, according to an accepted model of neural activation measurement, as posture changes. The disclosed devices and methods make use of a plurality of predetermined reference activation plots that have been measured for the patient in respective postures preferably covering the full range of postures likely to be assumed by the patient. The disclosed methods and devices are configured to estimate a defining parameter of the activation plot corresponding to a current measurement of neural response intensity, and from that defining parameter, a measure of neural activation according to the accepted model may be obtained. That measure may then be used as the feedback variable in a conventional feedback controller to adjust the stimulus intensity to bring the measure of neural activation closer to a target. Alternatively, that measure of recruitment may be used to compute a target response intensity for a conventional feedback controller in which response intensity is the feedback variable.

According to one aspect of the present technology, there is provided an implantable neuromodulation device for controllably delivering neural stimuli, the device comprising: a stimulus source configured to provide neural stimuli to be delivered via one or more stimulus electrodes of an electrode array to a neural pathway of a patient in order to evoke a neural response from the neural pathway; measurement circuitry configured to capture signal windows from signals sensed on the neural pathway via one or more measurement electrodes of the electrode array subsequent to respective neural stimuli; and a control unit configured to: control the stimulus source to provide a neural stimulus according to a stimulus intensity parameter; measure an intensity of an evoked neural response in a captured signal window subsequent to the neural stimulus; estimate one or more defining parameters of an intermediate activation plot corresponding to a current posture of the patient from the measured intensity of the evoked neural response, the stimulus intensity parameter, and a plurality of reference defining parameters of activation plots corresponding to respective reference postures; and adjust, using the one or more estimated defining parameters, the stimulus intensity parameter so as to maintain a recruitment value of the provided neural stimuli at or near a recruitment target value.

According to a second aspect of the present technology, there is provided an automated method of controllably delivering neural stimuli to a neural pathway of a patient, the method comprising: delivering a neural stimulus to the neural pathway of the patient in order to evoke a neural response from the neural pathway, the neural stimulus being delivered according to a stimulus intensity parameter; capturing a signal window sensed on the neural pathway subsequent to the delivered neural stimulus; measuring an intensity of a neural response evoked by the delivered neural stimulus in the captured signal window; estimating one or more defining parameters of an intermediate activation plot corresponding to a current posture of the patient from the measured intensity of the evoked neural response, the stimulus intensity parameter, and a plurality of reference defining parameters of activation plots corresponding to respective reference postures; and adjusting, using the one or more estimated defining parameters, the stimulus intensity parameter so as to maintain a recruitment value of the delivered neural stimuli at or near a recruitment target value.

According to a third aspect of the present technology, there is provided a neural stimulation system comprising: an implantable neuromodulation device for controllably delivering neural stimuli, the implantable neuromodulation device comprising: a stimulus source configured to provide neural stimuli to be delivered via one or more stimulus electrodes of an electrode array to a neural pathway of a patient in order to evoke a neural response from the neural pathway; measurement circuitry configured to capture signal windows sensed on the neural pathway via one or more sense electrodes of the electrode array subsequent to respective neural stimuli; and a control unit configured to control the stimulus source to provide each neural stimulus according to a stimulus intensity parameter; a processor configured to: instruct the control unit to control the stimulus source to provide a neural stimulus according to a stimulus intensity parameter; measure an intensity of an evoked neural response in the captured signal window subsequent to the provided neural stimulus; estimate one or more defining parameters of an intermediate activation plot corresponding to a current posture of the patient from the measured intensity of the evoked neural response, the stimulus intensity parameter, and a plurality of reference defining parameters of reference activation plots corresponding to respective reference postures; and adjust, using the one or more estimated defining parameters, the stimulus intensity parameter so as to maintain a recruitment value of the provided neural stimuli at or near a recruitment target value.

According to a fourth aspect of the present technology, there is provided a closed-loop neural stimulation device for controllably delivering neural stimuli, the device comprising: a control unit configured to repeatedly adjust a stimulus intensity parameter of provided neural stimuli so as to maintain a recruitment value of the provided neural stimuli at or near a recruitment target value, wherein the control unit is configured to adjust the stimulus intensity parameter by: estimating one or more defining parameters of an intermediate activation plot corresponding to a current posture from: a measured intensity of an evoked neural response to a delivered neural stimulus, a stimulus intensity parameter of the delivered neural stimulus, and a plurality of reference defining parameters of activation plots corresponding to respective reference postures; and adjusting, using the one or more estimated defining parameters, the stimulus intensity parameter so as to move the recruitment value closer to the recruitment target value.

According to a fifth aspect of the present technology, there is provided a neural stimulation system comprising: an implantable neuromodulation device for controllably delivering neural stimuli, the device comprising: a stimulus source configured to provide neural stimuli to be delivered via one or more stimulus electrodes of an electrode array to a neural pathway of a patient in order to evoke a neural response from the neural pathway; measurement circuitry configured to capture signal windows sensed on the neural pathway via one or more measurement electrodes of the electrode array subsequent to respective neural stimuli; and a control unit configured to: control the stimulus source to provide a neural stimulus according to a stimulus intensity parameter; and measure an intensity of an evoked neural response in the captured signal window subsequent to the provided neural stimulus; and a processor configured to iteratively: instruct the control unit to control the stimulus source to provide neural stimuli according to a plurality of different stimulus intensity parameters; instruct the control unit to measure respective intensities of neural responses in the captured signal windows subsequent to the respective provided neural stimuli; and estimate a defining parameter of a reference activation plot from the measured intensities and the respective stimulus intensity parameters; to obtain a first defining parameter of a first reference activation plot and a second defining parameter of a second reference activation plot; determine a metric from the first defining parameter and the second defining parameter; and determine, from the metric, an indication that a constant-recruitment closed-loop neural stimulation system is suitable for the patient.

According to a sixth aspect of the present technology, there is provided An automated method of controllably delivering neural stimuli to a neural pathway of a patient, the method comprising: iteratively: (i) delivering neural stimuli to the neural pathway of the patient in order to evoke neural responses from the neural pathway, each neural stimulus being delivered according to a corresponding stimulus intensity parameter; (ii) capturing signal windows sensed on the neural pathway subsequent to the respective delivered neural stimuli; (iii) measuring respective intensities of neural responses evoked by respective neural stimuli in the captured signal windows; and (iv) estimating a defining parameter of a reference activation plot from the measured intensities and the respective stimulus intensity parameters; to obtain a first defining parameter of a first reference activation plot and a second defining parameter of a second reference activation plot; determining a metric from the first defining parameter and the second defining parameter; and determining, from the metric, an indication that a constant-recruitment closed-loop neural stimulation system is suitable for the patient.

Related to the present technology is an implantable neuromodulation device for controllably delivering neural stimuli. The device comprises: a plurality of electrodes including one or more stimulus electrodes and one or more sense electrodes; a stimulus source configured to provide neural stimuli to be delivered via the one or more stimulus electrodes to a neural pathway of a patient in order to evoke a neural response from the neural pathway; measurement circuitry configured to capture signal windows sensed on the neural pathway via the one or more sense electrodes subsequent to respective neural stimuli; and a control unit. The control unit is configured to: control the stimulus source to provide a neural stimulus according to a stimulus intensity parameter; measure an intensity of an evoked neural response in the captured signal window subsequent to the provided neural stimulus; estimate one or more key parameters of an intermediate activation plot corresponding to a current posture of the patient from the measured intensity of the evoked neural response, the stimulus intensity parameter, and a plurality of key parameters of a respective plurality of reference activation plots corresponding to respective reference postures; and implement a feedback controller configured to use the one or more estimated key parameters to control the stimulus intensity parameter so as to maintain a recruitment value of the provided neural stimuli at or near a recruitment target value.

Related to the present technology is an automated method of controllably delivering neural stimuli to a neural pathway of a patient. The method comprises: delivering a neural stimulus to the neural pathway of the patient in order to evoke a neural response from the neural pathway, the neural stimulus being delivered according to a stimulus intensity parameter; capturing a signal window sensed on the neural pathway subsequent to the delivered neural stimulus; measuring an intensity of a neural response evoked by the delivered neural stimulus in the captured signal window; estimating a value of neural recruitment from the measured intensity of the evoked neural response, the stimulus intensity parameter, and a plurality of key parameters of a respective plurality of activation plots corresponding to respective reference postures; estimating one or more key parameters of an intermediate activation plot corresponding to a current posture of the patient from the measured intensity of the evoked neural response, the stimulus intensity parameter, and a plurality of key parameters of a respective plurality of activation plots corresponding to respective reference postures; and implementing a feedback controller configured to use the one or more estimated key parameters to control the stimulus intensity parameter so as to maintain a recruitment value of the delivered neural stimuli at or near a recruitment target value.

Related to the present technology is a neural stimulation system comprising an implantable neuromodulation device for controllably delivering neural stimuli, and a processor. The device comprises: a plurality of electrodes including one or more stimulus electrodes and one or more sense electrodes; a stimulus source configured to provide neural stimuli to be delivered via the one or more stimulus electrodes to a neural pathway of a patient in order to evoke a neural response from the neural pathway; measurement circuitry configured to capture signal windows sensed on the neural pathway via the one or more sense electrodes subsequent to respective neural stimuli; and a control unit configured to control the stimulus source to provide each neural stimulus according to a stimulus intensity parameter. The processor is configured to: instruct the control unit to control the stimulus source to provide a neural stimulus according to a stimulus intensity parameter; measure an intensity of an evoked neural response in the captured signal window subsequent to the provided neural stimulus; estimate one or more key parameters of an intermediate activation plot corresponding to a current posture of the patient from the measured intensity of the evoked neural response, the stimulus intensity parameter, and a plurality of key parameters of a respective plurality of reference activation plots corresponding to respective reference postures; and implement a feedback controller configured to use the one or more estimated key parameters to control the stimulus intensity parameter so as to maintain a recruitment value of the provided neural stimulus at or near a recruitment target value.

Related to the present technology is a neural stimulation system comprising an implantable neuromodulation device for controllably delivering neural stimuli, and a processor. The device comprises: a plurality of electrodes including one or more stimulus electrodes and one or more sense electrodes; a stimulus source configured to provide neural stimuli to be delivered via the one or more stimulus electrodes to a neural pathway of a patient in order to evoke a neural response from the neural pathway; measurement circuitry configured to capture signal windows sensed on the neural pathway via the one or more sense electrodes subsequent to respective neural stimuli; and a control unit configured to: control the stimulus source to provide a neural stimulus according to a stimulus intensity parameter; and measure an intensity of an evoked neural response in the captured signal window subsequent to the provided neural stimulus. The processor is configured to: instruct the control unit to control the stimulus source to provide neural stimuli according to a plurality of different stimulus intensity parameters; instruct the control unit to measure respective intensities of neural responses in the captured signal windows subsequent to the respective provided neural stimuli; estimate a first key parameter of a first activation plot from the measured intensities and the respective stimulus intensity parameters; repeat the first instructing, second instructing, and estimating to obtain a second key parameter of a second activation plot; compute a metric from the first key parameter and the second key parameter; and determine, from the computed metric, an indication that a constant-recruitment closed-loop neural stimulation system is suitable for the patient.

Related to the present technology is an automated method of controllably delivering neural stimuli to a neural pathway of a patient. The method comprises: delivering neural stimuli to the neural pathway of the patient in order to evoke neural responses from the neural pathway, each neural stimulus being delivered according to a corresponding stimulus intensity parameter; capturing signal windows sensed on the neural pathway subsequent to the respective delivered neural stimuli; measuring respective intensities of neural responses in the captured signal windows; estimating a first key parameter of a first activation plot from the measured intensities and the respective stimulus intensity parameters; repeating the delivering, capturing, measuring, and estimating to obtain a second key parameter of a second activation plot; computing a metric from the first key parameter and the second key parameter; and determining, from the computed metric, an indication that a constant-recruitment closed-loop neural stimulation system is suitable for the patient.

References herein to estimation, determination, comparison and the like are to be understood as referring to an automated process carried out on data by a processor operating to execute a predefined procedure suitable to effect the described estimation, determination or comparison step(s).

The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be embodied as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored or executed in a distributed fashion.

### BRIEF DESCRIPTION OF THE DRAWINGS

Notwithstanding any other implementations which may fall within the scope of the present invention, implementations of the invention will now be described, by way of example only, with reference to the accompanying drawings, in which:

FIG. 1 schematically illustrates an implanted spinal cord stimulator, according to one implementation of the present technology;

FIG. 2 is a block diagram of the stimulator of FIG. 1;

FIG. 3 is a schematic illustrating interaction of the implanted stimulator of FIG. 1 with a nerve;

FIG. 4a illustrates an idealised activation plot for one posture of a patient undergoing neural stimulation;

FIG. 4b illustrates the variation in the activation plots with changing posture of the patient;

FIG. 5 is a schematic illustrating elements and inputs of a closed-loop neural stimulation (CLNS) system, according to one implementation of the present technology;

FIG. 6 illustrates the typical form of an electrically evoked compound action potential (ECAP) of a healthy subject;

FIG. 7 is a graph containing activation plots for a patient in a supine posture, a standing posture and a sitting posture, respectively;

FIG. 8 is a graph in IT-VI space showing a point representing an activation plot (AP) such as the AP of FIG. 4a;

FIG. 9a is a graph in I-V space illustrating two reference APs that have been previously constructed for the least-sensitive and most-sensitive reference postures respectively;

FIG. 9b is a graph in IT-VI space on which are marked points representing the APs AP1 and AP2 from FIG. 9a;

FIG. 10a is a graph in I-V space showing AP1 and AP2 as in FIG. 9a;

FIG. 10b is a graph in IT-VI space containing the points representing AP1 and AP2, as in FIG. 9b;

FIG. 11 is a graph illustrating the computation of an interpolation fraction according to one implementation;

FIGS. 12a and 12b are graphs illustrating a method of interpolation that makes use of the interpolation fraction of FIG. 11 according to one implementation of the present technology;

FIGS. 13a and 13b are graphs illustrating a method of interpolation that makes use of the interpolation fraction of FIG. 11 according to one implementation of the present technology;

FIGS. 14a and 14b are graphs illustrating another method of interpolation according to another implementation of the present technology;

FIG. 15 is a schematic illustrating elements and inputs of a constant-recruitment CLNS system, according to one implementation of the present technology;

FIG. 16 is a flow chart illustrating a method 1600 of determining a suitable feedback controller for a patient; and

FIG. 17 is a schematic illustrating elements and inputs of a constant-recruitment CLNS system, according to another implementation of the present technology.

### DETAILED DESCRIPTION OF THE PRESENT TECHNOLOGY

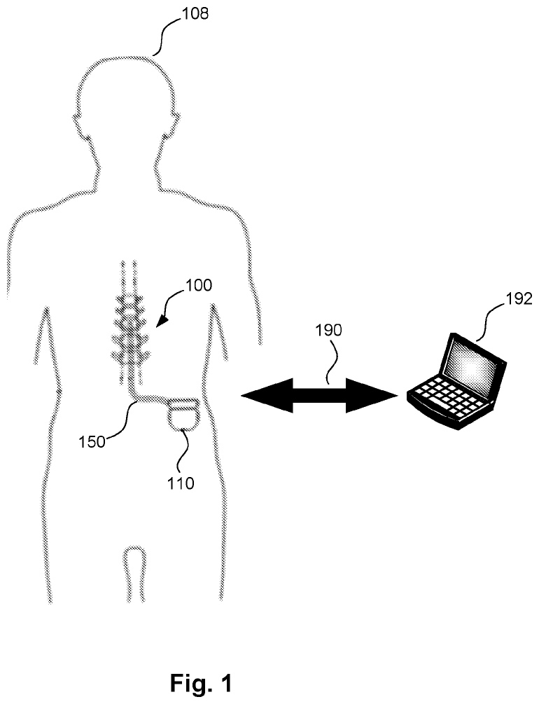

FIG. 1 schematically illustrates an implanted spinal cord stimulator 100 in a patient 108, according to one implementation of the present technology. Stimulator 100 comprises an electronics module 110 housed within a conductive case, implanted at a suitable location. In one implementation, stimulator 100 is implanted in the patient's lower abdominal area or posterior superior gluteal region. In other implementations, the electronics module 110 is implanted in other locations, such as in a flank or sub-clavicularly. The electronics module 110 is configured to electrically connect to an electrode assembly comprising an electrode array 150 implanted within the epidural space and connected to the module 110 by a suitable lead. The electrode array 150 may comprise one or more electrodes such as electrode pads on a paddle lead, circular (e.g., ring) electrodes surrounding the body of the lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus be used as a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

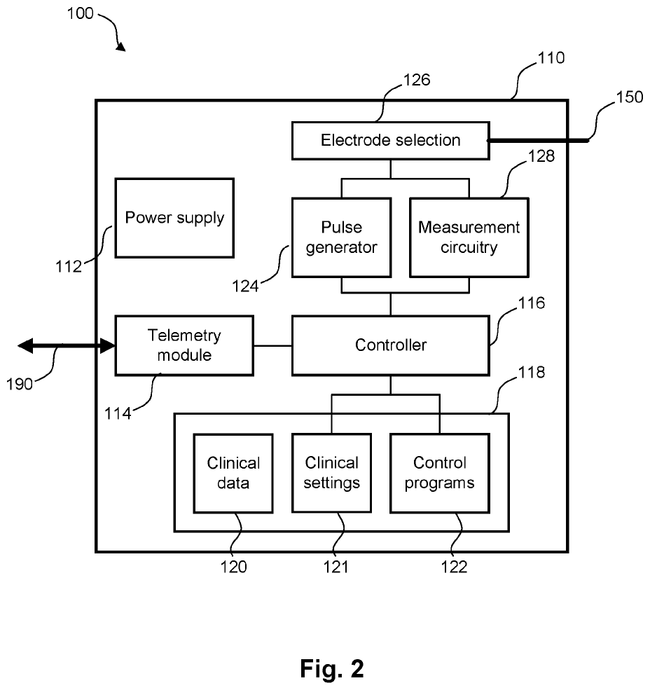

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive or inductive transfer, may be used by telemetry module 114 to transfer power or data to and from the electronics module 110 via communications channel 190. Module controller 116 has an associated memory 118 storing one or more of clinical data 120, clinical settings 121, control programs 122, and the like. Controller 116 is configured by control programs 122, sometimes referred to as firmware, to control a pulse generator 124 to generate stimuli, such as in the form of electrical pulses, in accordance with the clinical settings 121. Electrode selection module 126 switches the generated pulses to the selected electrode(s) of electrode array 150, for delivery of the pulses to the tissue surrounding the selected electrode(s). Measurement circuitry 128, which may comprise an amplifier or an analog-to-digital converter (ADC), is configured to process signals comprising neural responses sensed at measurement electrode(s) of the electrode array 150 as selected by electrode selection module 126.

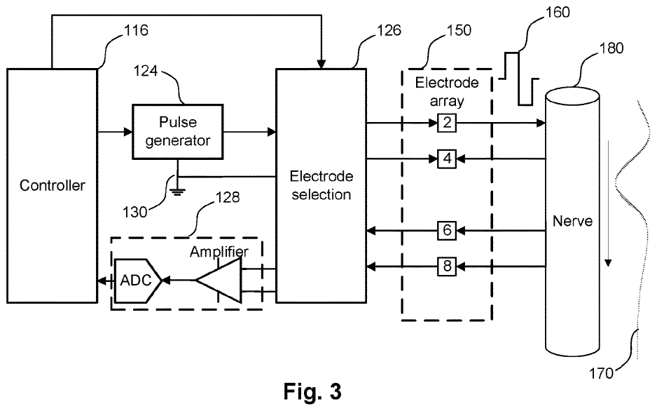

FIG. 3 is a schematic illustrating interaction of the implanted stimulator 100 with anerve 180 in the patient 108. In the implementation illustrated in FIG. 3 the nerve 180 may be located in the spinal cord, however in alternative implementations the stimulator 100 may be positioned adjacent any desired neural tissue including a peripheral nerve, visceral nerve, parasympathetic nerve or a brain structure. Electrode selection module 126 selects a stimulus electrode 2 of electrode array 150 through which to deliver a pulse from the pulse generator 124 to surrounding tissue including nerve 180. A pulse may comprise one or more phases, e.g. a monophasic pulse comprises one phase, and a biphasic stimulus pulse 160 comprises two phases. Electrode selection module 126 also selects a return electrode 4 of the electrode array 150 for stimulus current return in each phase, to maintain a zero net charge transfer. An electrode may act as both a stimulus electrode and a return electrode over a complete multiphasic stimulus pulse. The use of two electrodes in this manner for delivering and returning current in each stimulus phase is referred to as bipolar stimulation. Alternative implementations may apply other forms of bipolar stimulation, or may use a greater number of stimulus or return electrodes. By contrast, in monopolar stimulation, current is returned through the conductive case of the stimulator 100, which may therefore be configured and function as an electrode though it is not physically part of the electrode array 150. The set of stimulus electrodes and return electrodes is referred to as the stimulus electrode configuration. Electrode selection module 126 is illustrated as connecting to a ground 130 of the pulse generator 124 to enable stimulus current return via the return electrode 4. However, other connections for current return may be used in other implementations.

Delivery of an appropriate stimulus via electrodes 2 and 4 to the nerve 180 evokes a neural response 170 comprising an evoked compound action potential (ECAP) which will propagate along the nerve 180 as illustrated at a rate known as the conduction velocity. The ECAP may be evoked for therapeutic purposes, which in the case of a spinal cord stimulator for chronic pain may be to create paraesthesia at a desired location. To this end, the electrodes 2 and 4 are used to deliver stimuli periodically at any therapeutically suitable stimulus frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that is experienced by the user as paraesthesia. When a stimulus electrode configuration is found which evokes paraesthesia in a location and of a size which is congruent with the area of the patient's body affected by pain and of a quality that is comfortable for the patient, the clinician or the patient nominates that configuration for ongoing use. The therapy parameters may be loaded into the memory 118 of the stimulator 100 as the clinical settings 121.

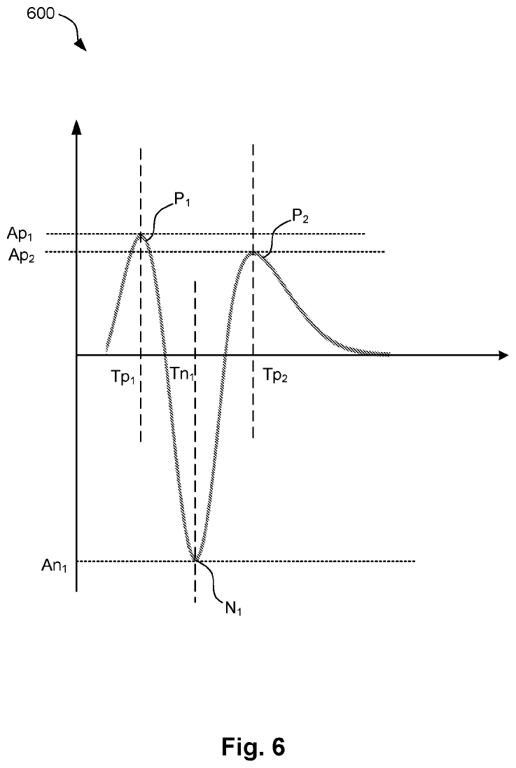

FIG. 6 illustrates the typical form of an ECAP 600 of a healthy subject, as recorded at a single measurement electrode referenced to the system ground 130. The shape and duration of the single-ended ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Differential ECAP measurements are less subject to common-mode noise on the surrounding tissue than single-ended ECAP measurements. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be characterised by any suitable characteristic(s) of which some are indicated in FIG. 6. The amplitude of the positive peak P1 is Ap₁ and occurs at time Tp₁. The amplitude of the positive peak P2 is Ap₂ and occurs at time Tp₂. The amplitude of the negative peak P1 is An₁ and occurs at time Tn₁. The peak-to-peak amplitude is Ap₁+An₁. A recorded ECAP will typically have a maximum peak-to-peak amplitude in the range of microvolts and a duration of 2 to 3 ms.

The stimulator 100 is further configured to measure the intensity of ECAPs 170 propagating along nerve 180, whether such ECAPs are evoked by the stimulus from electrodes 2 and 4, or otherwise evoked. To this end, any electrodes of the array 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The recording electrode and the reference electrode are referred to as the measurement electrode configuration. The measurement circuitry 128 for example may operate in accordance with the teachings of the above-mentioned International Patent Publication No. WO2012/155183.

Signals sensed by the measurement electrodes 6, 8 and processed by measurement circuitry 128 are further processed by an ECAP detector implemented within controller 116, configured by control programs 122, to obtain information regarding the effect of the applied stimulus upon the nerve 180. In some implementations, the sensed signals are processed by the ECAP detector in a manner which measures and stores one or more characteristics from each evoked neural response or group of evoked neural responses contained in the sensed signal. In one such implementation, the characteristic comprises a peak-to-peak ECAP amplitude in microvolts (pV). For example, the sensed signals may be processed by the ECAP detector to determine the peak-to-peak ECAP amplitude in accordance with the teachings of International Patent Publication No. WO2015/074121, the contents of which are incorporated herein by reference. Alternative implementations of the ECAP detector may measure and store an alternative characteristic from the neural response, or may measure and store two or more characteristics from the neural response.

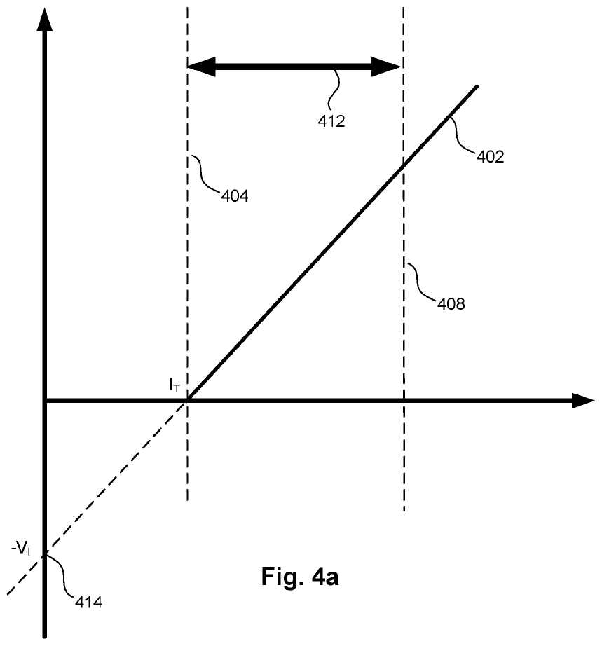

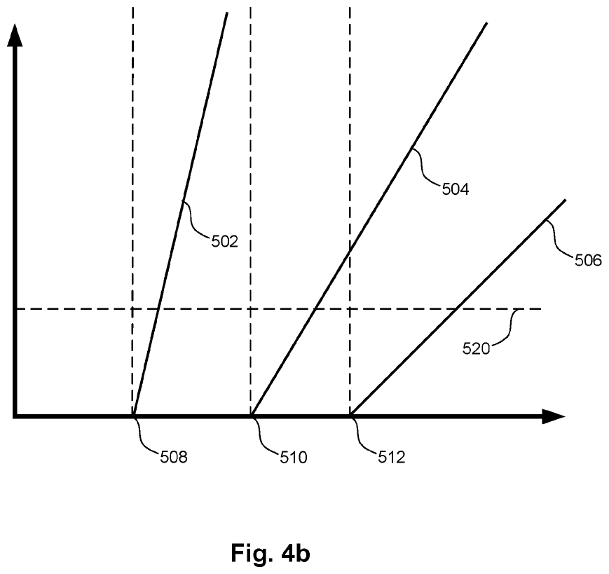

An activation plot, or growth curve, is an approximation to the relationship between stimulus intensity (e.g. an amplitude of the current pulse 160) and intensity of neural response 170 evoked by the stimulus (e.g. an ECAP amplitude). FIG. 4a illustrates an idealised activation plot 402 for one posture of the patient 108. The activation plot 402 shows a linearly increasing ECAP amplitude for stimulus intensity values above a threshold 404 referred to as the recruitment threshold, or simply as the threshold. The recruitment threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The recruitment threshold 404 therefore reflects the field strength at which significant numbers of fibres begin to be recruited, and the increase in response intensity with stimulus intensity above the recruitment threshold reflects increasing numbers of fibres being recruited. Below the recruitment threshold 404, the response intensity may be taken to be zero. Above the recruitment threshold 404, the activation plot 402 has a positive, approximately constant slope indicating a linear relationship between stimulus intensity and the response intensity. Such a relationship may be modelled as:

$$V = \{ \begin{matrix}S ( I - {I}_{T} ) , & I \geq {I}_{T} \\ 0 , & I < {I}_{T}\end{matrix} \qquad ( 1 )$$

where I is the stimulus intensity, V is the response intensity, IT is the recruitment threshold 404 and S is the slope of the activation plot (referred to herein as the patient sensitivity). The sensitivity S and the recruitment threshold IT are the defining parameters of the activation plot 402 according to the model of Equation (1).

As illustrated in FIG. 4a, the notional extension of the activation plot 402 below the stimulus intensity axis intercepts the response intensity axis at V=−V_{I} (the point 414). The value V_{I} is referred to herein as the response intercept. An alternative, equivalent model for the relationship between stimulus intensity and the response intensity is as follows:

$$V = \{ \begin{matrix}\frac{{V}_{I}}{{I}_{T}} ( I - {I}_{T} ) , & I \geq {I}_{T} \\ 0 , & I < {I}_{T}\end{matrix} \qquad ( 2 )$$

where V_{I} is the response intercept. The response intercept (or more succinctly, the intercept) V_{I} and the recruitment threshold (or more succinctly, the threshold) IT are the defining parameters of the activation plot 402 according to the alternative model of Equation (2).

FIG. 4a also illustrates a discomfort threshold 408, which is a stimulus intensity above which the patient 108 experiences uncomfortable or painful stimulation. For effective and comfortable operation of an implantable neuromodulation device such as the stimulator 100, it is desirable to maintain stimulus intensity within a therapeutic range. A stimulus intensity within a therapeutic range 412 is above the recruitment threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

FIG. 4b illustrates the variation in the activation plots with changing posture of the patient. A change in posture of the patient may cause a change in impedance of the electrode-tissue interface or a change in the distance between electrodes and the spinal cord. Electrode-to-cord distance is therefore loosely referred to throughout the present disclosure as posture. While the activation plots for only three postures, 502, 504 and 506, are shown in FIG. 4b, the activation plot for any given posture can lie between or outside the activation plots shown, on a continuously varying basis depending on posture. Consequently, as the patient's posture changes, the recruitment threshold changes, as indicated by the recruitment thresholds 508, 510, and 512 for the respective activation plots 502, 504, and 506. Additionally, as the patient's posture changes, the patient sensitivity also changes, as indicated by the varying slopes of activation plots 502, 504, and 506. In general, as the distance between the stimulus electrodes and the spinal cord increases, the recruitment threshold increases and the sensitivity decreases. The activation plots 502, 504, and 506 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus intensity based on a feedback variable that is determined from one or more measured ECAP characteristics. In one implementation, the device may adjust the stimulus intensity to maintain the measured ECAP amplitude at a target response intensity. In other words, the feedback variable is the measured ECAP amplitude. For example, the device may calculate an error between a target ECAP amplitude and a measured ECAP amplitude, and adjust the applied stimulus intensity to reduce the error as much as possible, such as by adding the scaled error to the current stimulus intensity. A neuromodulation device that operates by adjusting the applied stimulus intensity based on a measured ECAP characteristic is said to be operating in closed-loop mode and will also be referred to as a closed-loop neural stimulation (CLNS) device. By adjusting the applied stimulus intensity to maintain the measured ECAP amplitude at an appropriate target response intensity, such as a target ECAP amplitude 520 illustrated in FIG. 4b, a CLNS device will generally keep the stimulus intensity within the therapeutic range as patient posture varies.

A CLNS device comprises a stimulator that takes a stimulus intensity value and converts it into a neural stimulus comprising a sequence of electrical pulses according to a predefined stimulation pattern. The stimulation pattern is parametrised by multiple stimulus parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the feedback loop.

In an example CLNS system, a user (e.g. the patient or a clinician) sets a target response intensity, and the CLNS device performs proportional-integral-differential (PID) control. In some implementations, the differential contribution is disregarded and the CLNS device uses a first order integrating feedback loop. The stimulator produces a stimulus in accordance with a stimulus intensity parameter, which evokes a neural response in the patient. The intensity of an evoked neural response (e.g. an ECAP) is measured by the CLNS device and compared to the target response intensity.

The measured neural response intensity, and its deviation from the target response intensity, is used by the feedback loop to determine possible adjustments to the stimulus intensity parameter to maintain the neural response at the target intensity. If the target intensity is properly chosen, the patient receives consistently comfortable and therapeutic stimulation through posture changes and other perturbations to the stimulus/response behaviour.

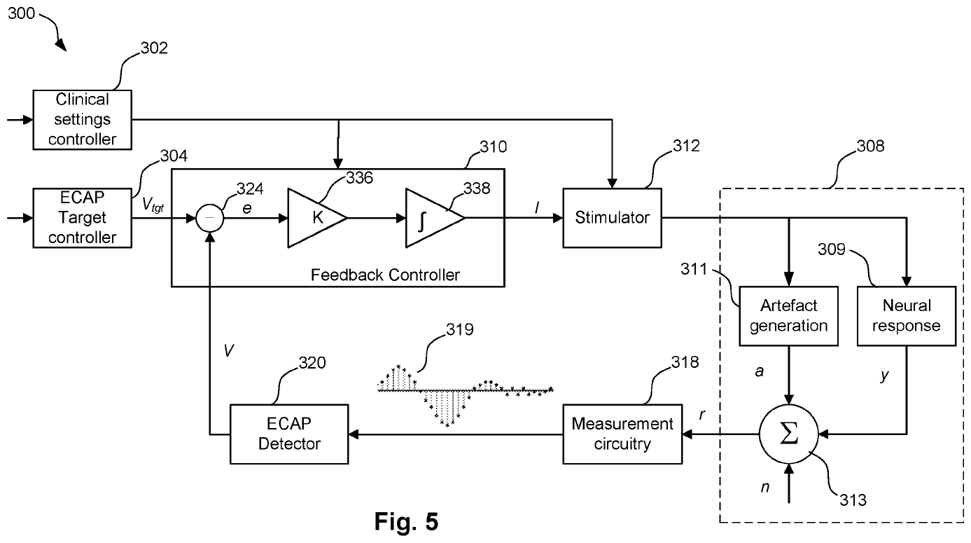

FIG. 5 is a schematic illustrating elements and inputs of a closed-loop neural stimulation (CLNS) system 300, according to one implementation of the present technology. The system 300 comprises a stimulator 312 which converts a stimulus intensity parameter (for example a stimulus current amplitude) s, in concert with a set of predefined stimulus parameters, to a neural stimulus comprising a sequence of electrical pulses on the stimulus electrodes (not shown in FIG. 5). According to one implementation, the predefined stimulus parameters comprise the number and order of phases, the number of stimulus electrode poles, the pulse width, and the stimulus rate or frequency.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrodes. Various sources of measurement noise n, as well as the artefact a, may add to the evoked response y at the summing element 313 to form the sensed signal r, including: electrical noise from external sources such as 50 Hz mains power; electrical disturbances produced by the body such as neural responses evoked not by the device but by other causes such as peripheral sensory input; EEG; EMG; and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (including evoked neural response, artefact, and measurement noise), and samples the amplified sensed signal r to capture a “signal window” 319 comprising a predetermined number of samples of the amplified sensed signal r. The ECAP detector 320 processes the signal window 319 and outputs a measured neural response intensity V. In one implementation, the neural response intensity comprises a peak-to-peak ECAP amplitude. The measured response intensity V(an example of a feedback variable) is input into the feedback controller 310. The feedback controller 310 comprises a comparator 324 that compares the measured response intensity V to a target response intensity V_{tgt} as set by the ECAP target controller 304 and provides an indication of the difference between the measured response intensity V and the target response intensity V_{tgt}. This difference is the error value, e.

The feedback controller 310 calculates an adjusted stimulus intensity parameter I with the aim of maintaining a measured response intensity V at or near the target response intensity V_{tgt}. Accordingly, the feedback controller 310 adjusts the stimulus intensity parameter I to minimise the error value, e. In one implementation, the controller 310 utilises a first order integrating function, using a gain element 336 and an integrator 338, in order to provide suitable adjustment to the stimulus intensity parameter I. According to such an implementation, the stimulus intensity parameter I may be determined by the feedback controller 310 as

I=∫Ke·dt (3)
where K is the gain of the gain element 336 (the controller gain). This relation may also be represented as

δI=Ke (4)
where δI is an adjustment to the stimulus intensity parameter I.

A target response intensity V_{tgt} is input to the feedback controller 310 via the ECAP target controller 304. In one implementation, the ECAP target controller 304 provides an indication of a specific target response intensity. In another implementation, the ECAP target controller 304 provides an indication to increase or to decrease the present target response intensity. The ECAP target controller 304 may comprise an input into the CLNS system 300, via which the patient or clinician can input a target response intensity, or indication thereof. The ECAP target controller 304 may comprise memory in which the target response intensity is stored, and from which the target response intensity is provided to the feedback controller 310.

A clinical settings controller 302 provides clinical settings to the system 300, including the feedback controller 310 and the stimulus parameters for the stimulator 312 that are not under the control of the feedback controller 310. In one example, the clinical settings controller 302 may be configured to adjust the controller gain K of the feedback controller 310 to adapt the feedback loop to patient sensitivity. The clinical settings controller 302 may comprise an input into the CLNS system 300, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 16 kHz). As the ECAP detector 320 is linear, only the stimulus clock affects the dynamics of the CLNS system 300. On the next stimulus clock cycle, the stimulator 312 outputs a stimulus in accordance with the adjusted stimulus intensity I. Accordingly, there is a delay of one stimulus clock cycle before the stimulus intensity is updated in light of the error value e.

### A Measure of Neural Recruitment

Neural recruitment refers to the number of fibres recruited by a stimulus provided by a neural stimulator. The stimulus current, flowing through the one or more stimulus electrodes to the one or more return electrodes, generates a field at the neural tissue (e.g. the spinal cord). It will be appreciated that for a given stimulus intensity, as the stimulus electrode changes distance with respect to the cord, the field intensity at the neural tissue changes. The stimulus intensity at which the field generated by the stimulus begins to recruit fibres, for a specific posture of a patient, is the recruitment threshold for that posture of the patient.

The recruitment of fibres begins at the same field intensity at the spinal cord regardless of the patient's posture. Therefore, the recruitment threshold for a patient's current posture provides a calibration point for estimating the amount of recruitment of fibres resulting from stimuli at a given stimulus intensity. At stimulus intensities above the recruitment threshold, the recruitment of the field generated by the stimulus increases with the ratio by which the stimulus current exceeds the threshold stimulus current. The measure R of recruitment of fibres resulting from stimuli at a given stimulus intensity I may therefore be written as follows:

$$R = \frac{I}{{I}_{T}} - 1 \qquad ( 5 )$$

where I_{T} is the recruitment threshold for the patient's current posture.

Using Equation (2), the measure R of recruitment of fibres when the evoked neural response intensity is V may equivalently be estimated as follows:

$$R = \frac{V}{{V}_{I}} \qquad ( 6 )$$

where V_{I} is the response intercept for the patient's current posture.

In other words, the recruitment may be characterized as the excess of the stimulus intensity over the recruitment threshold, normalised by the recruitment threshold, or as the response intensity normalised by the response intercept.

If the patient moves into a less sensitive posture at a fixed stimulus intensity, the neural recruitment decreases, and the measured ECAP amplitude decreases. If the patient moves into a more sensitive posture, the neural recruitment increases, and the measured ECAP amplitude increases.

Although there may be alignment between the variation in ECAP amplitude and the variation in neural recruitment due to posture changes, the relationship between these two measures is not proportional in all situations.

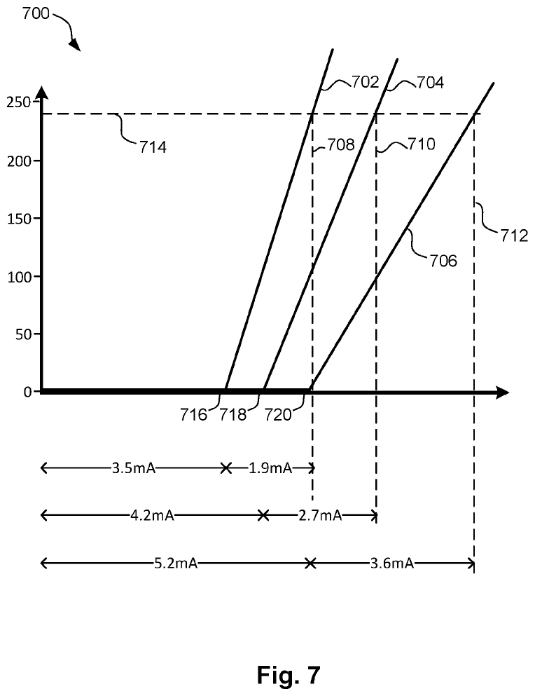

FIG. 7 is a graph 700 containing activation plots 702, 704 and 706, for a patient in a supine posture, a standing posture and a sitting posture, respectively. Dashed line 714 indicates a measured ECAP amplitude of 240 μV.

The activation plot 702 for the supine (most sensitive) posture has a recruitment threshold 716 at 3.5 mA, and has a measured ECAP value of 240 μV when the stimulus current is 5.4 mA, as indicated by dashed line 708. Applying Equation (5), recruitment at a measured ECAP value of 240 μV, when the patient is in the supine posture, is equal to 0.54.

The activation plot 704 for the standing posture has a recruitment threshold 718 at 4.2 mA, and has a measured ECAP value of 240 μV when the stimulus current is 6.9 mA, as indicated by dashed line 710. Applying Equation (5), recruitment at a measured ECAP value of 240 μV, when the patient is in the standing posture, is equal to 0.64.

The activation plot 706 for the sitting (least sensitive) posture has a recruitment threshold 720 at 5.2 mA, and has a measured ECAP value of 240 μV when the stimulus current is 8.8 mA, as indicated by dashed line 712. Applying Equation (5), recruitment at a measured ECAP value of 240 μV, when the patient is in the sitting posture, is equal to 0.69.

Accordingly, for the same ECAP value (e.g. 240 μV in the example of FIG. 7) the recruitment level of the fibres varies from 0.54 to 0.64 to 0.69 as the patient moves from one, most sensitive, posture to a second and a third, least sensitive, posture. In the example of FIG. 7, the recruitment level of the fibres increases as the patient moves from the supine (most sensitive) posture, to the standing posture, and then to the sitting (least sensitive) posture.

Accordingly, a CLNS system 300 that is configured to maintain a target ECAP amplitude (a constant-ECAP CLNS system) may result in the patient perceiving more intense stimulation, in the form of a higher level of recruitment, for the same ECAP amplitude, as the patient moves from a more sensitive posture to a less sensitive posture. A more intense level of stimulation may be undesirable as it may exceed the patient's discomfort threshold.

Aspects of the present technology relate to methods for maintaining, and devices configured to maintain, constant neural recruitment, as estimated by Equation (5) or Equation (6), as patient posture varies. Maintaining constant neural recruitment, i.e. at a target recruitment level, as estimated according to Equation (5) or Equation (6), may lead to a constant level of neural stimulation, as perceived by the patient, and therefore a more consistent therapy as posture changes.

### Constant-Recruitment Feedback Control

A given stimulus intensity I and measured evoked response intensity Vin a given posture lie on an activation plot (AP) fitting the model given in Equation (2) for that posture, where the defining parameters of the AP (threshold IT and intercept V_{I}) are specific to that posture. If the threshold or the intercept of the AP were known, Equation (5) or Equation (6) could be utilised to estimate the recruitment R. The estimated recruitment R could then be used as a feedback variable in a feedback controller such as the feedback controller 310 of FIG. 5 to adjust the stimulus intensity I to maintain the estimated recruitment R at or near a recruitment target R_{tgt}, in the same way that the feedback controller 310 in the constant-ECAP CLNS system 300 of FIG. 5 adjusts the stimulus intensity I to maintain the neural response intensity V at or near a target response intensity V_{tgt}. However, the difficulty is that for any given stimulus intensity-evoked response intensity measurement pair (I, V), the current posture/AP/defining parameter(s) of the AP are unknown.

In implementations of a CLNS system according to the present technology, a defining parameter (the intercept or threshold) of the current AP is estimated from the current stimulus intensity-response intensity pair (I, V) and the defining parameters of reference APs previously measured for the patient in a plurality of reference postures. In some implementations, the reference postures cover the extremes of the range of sensitivities (i.e. electrode-cord distances), so that the estimation is an interpolation. For example, a most-sensitive reference posture may be supine, while a least-sensitive reference posture may be sitting. Each reference AP may be constructed, and its defining parameters estimated, by a clinical programming application (CPA) during a programming phase, as disclosed, for example, in International Patent Application no. PCT/AU2023/050356 by the present applicant, the content of which is hereby incorporated by reference. The defining parameters of each reference AP may be stored in the clinical settings 121 of the stimulator 100 by the CPA for later use by implementations of a CLNS system according to the present technology.

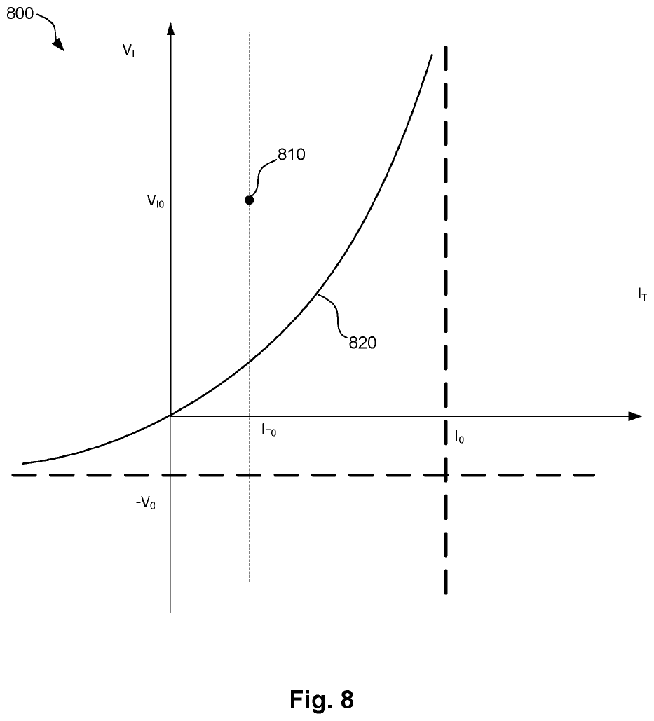

This process of interpolation is visualisable by representing an AP not as a line in I-V space, as the AP 402 is illustrated in FIG. 4a, but as a point in IT-V_{I} space. This is illustrated in FIG. 8, which shows a point 810 representing an AP such as the AP 402 on a graph 800 in IT-V_{I} space. The position of the point 810 along the horizontal (threshold) axis is the threshold I_{T0} for the AP, and the position of the point 810 along the vertical (intercept) axis is the intercept V_{I0} for the AP.

Also illustrated on the graph 800 in IT-V_{I} space is a curve 820 representing a stimulus intensity-response intensity pair (I₀, V₀). The expression for the curve 820 is obtained by rearranging Equation (2) so that the intercept V_{I} is the subject, as follows:

$${V}_{I} = \frac{{V}_{0} {I}_{T}}{{I}_{0} - {I}_{T}} \qquad ( 7 )$$

Each point on the graph 800 represents a unique AP (not all of which are physiologically meaningful). The curve 820 represents the set of all APs on which the point (I₀, V₀) lies in I-V space. It may be seen that the curve 820 approaches two asymptotes: the horizontal asymptote V_{I}=−V₀ as IT gets large negatively, and the vertical asymptote I_{T}=I₀ as V_{I} gets large positively. The curve 820 passes through the origin, since one of the APs on which the point (I₀, V₀) lies has a threshold I_{T} of zero and an intercept V_{I} of zero. However, the point 810 does not lie on the curve 820 as the AP with threshold I_{T0} and intercept V_{I0} does not include the point (I₀, V₀).

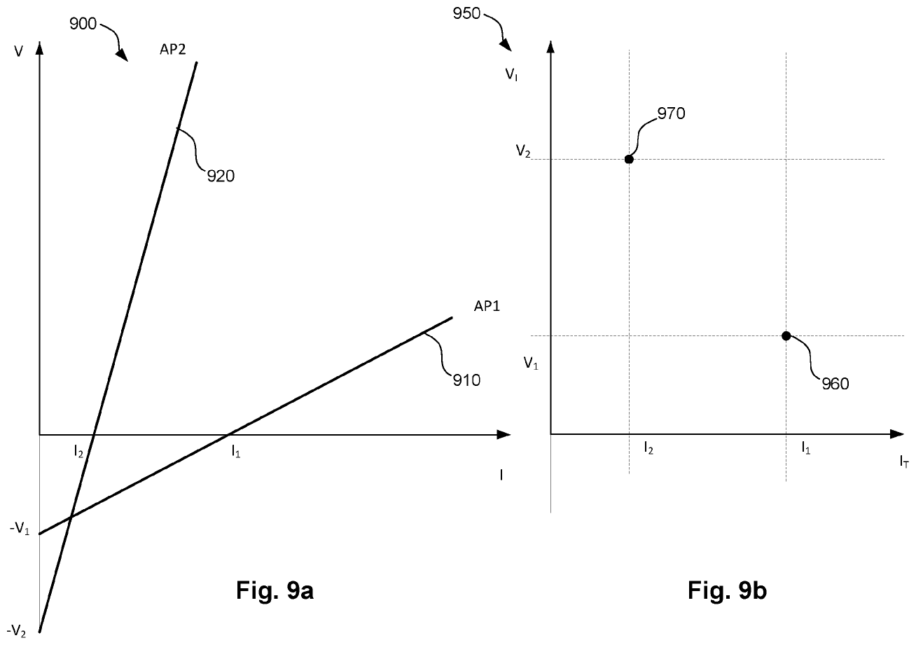

FIG. 9a is a graph 900 in I-V space illustrating two reference APs 910 and 920, referred to as AP1 and AP2 respectively, that have been previously constructed as described above for the least-sensitive and most-sensitive reference postures respectively. The thresholds for AP1 and AP2 are I1 and I2 respectively, while the intercepts are V1 and V2 respectively. FIG. 9b is a graph 950 in IT-VI space on which are marked the points 960 (I1, V1) and 970 (I2, V2) representing AP1 and AP2 respectively, just as the point 810 in FIG. 8 represents an AP with threshold I_{T0} and intercept V_{I0}.

The intercept ratio V2/V1 is the variation in recruitment R as defined in Equation (5) or Equation (6) that would be expected of a constant-ECAP CLNS system such as the CLNS system 300 that adjusted stimulus intensity I to maintain a constant response intensity V as posture changed from the least-sensitive (AP1) to the most-sensitive (AP2) posture, or vice versa. In the example of FIG. 7, the recruitment level of the fibres increased from 0.54 to 0.69 as the patient moved from the most sensitive posture to the least sensitive posture. The intercept ratio V2/V1 would therefore be 0.69/0.54=1.28 in the example of FIG. 7. In another example in which there was no change in recruitment R from maintaining a constant ECAP across postures, the intercept ratio V2/V1 would be equal to 1. The intercept ratio V2/V1 may therefore be used as a metric to determine whether a constant-ECAP CLNS system such as the CLNS system 300 is capable of maintaining constant recruitment with posture for the patient, or whether implementations of a constant-recruitment CLNS system according to the present technology are more suitable for maintaining constant recruitment with posture. For example, if the intercept ratio V2/V1 differs from 1 by more than a predetermined amount (for example 5 percent), implementations of a constant-recruitment CLNS system according to the present technology may be employed; otherwise, a constant-ECAP CLNS system such as the CLNS system 300 may be employed.

If a response intensity measurement of V₀ is obtained from a stimulus intensity of I₀ with the patient in an unknown, intermediate posture, the task is to interpolate a “sensible” AP, intermediate between AP1 and AP2, on which the point (I₀, V₀) lies. Under the reasonable assumption of monotonicity, such an intermediate AP will have threshold I_{T0} between I2 and I1 and intercept V_{I0} between V1 and V2. Finding either of these defining parameters of the intermediate AP including the point (I₀, V₀) will enable the use of Equation (5) or Equation (6) to estimate the recruitment R₀ obtained by the stimulus of intensity I₀.

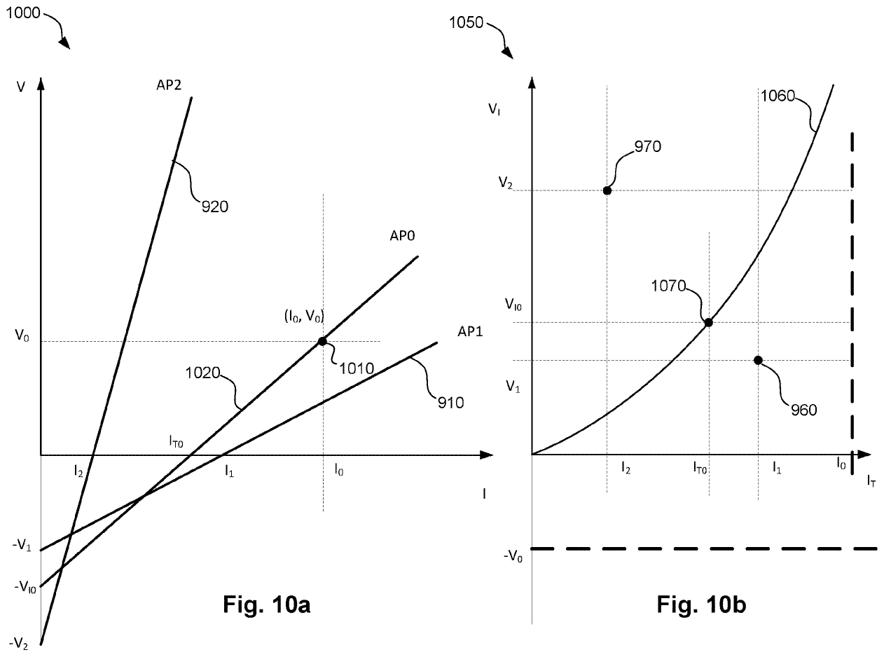

This situation is illustrated in FIG. 10a, which is a graph 1000 in I-V space showing AP1 (910) and AP2 (920) as in FIG. 9a. Also shown is the point (I₀, V₀) 1010, lying on the intermediate AP, labelled as AP0 (1020). AP0 1020 has a threshold I_{T0} between I2 and I1 and an intercept V_{I0} between V1 and V2.

FIG. 10b is an equivalent graph 1050 in IT-VI space. The graph 1050 contains the points 960 and 970 representing AP1 and AP2, as in FIG. 9b. The curve 1060 represents the point (I₀, V₀) 1010, just as the curve 820 in FIG. 8 represents the point (I₀, V₀). The interpolation between AP1 and AP2 to find AP0 on which the point (I₀, V₀) lies in I-V space is represented on the graph 1050 by an interpolation between the points 960 and 970 to find a point (I_{T0}, V_{I0}) 1070 on the curve 1060.

The interpolation between AP1 and AP2 to find AP0 in IT-VI space may be carried out in several ways.

In some implementations, an interpolation fraction a representing the location of the point (I₀, V₀) between AP1 and AP2 in I-V space may first be computed. FIG. 11 is a graph 1100 illustrating the computation of the fraction a according to one implementation, as a fractional horizontal distance between (I₀, V₀) and AP1. The stimulus intensity interval 1110 between the point where AP1 has response intensity V₀ and the point where AP2 has response intensity V₀ is partitioned into a sub-interval 1120 of fractional length a between AP1 and the point (I₀, V₀), and a sub-interval 1130 of fractional length 1-a between AP2 and the point (I₀, V₀). According to this implementation, the fraction a may be computed as follows:

$$a = \frac{{I}_{1} ( \frac{{V}_{0}}{{V}_{1}} + 1 ) - {I}_{0}}{{I}_{1} ( \frac{{V}_{0}}{{V}_{1}} + 1 ) - {I}_{2} ( \frac{{V}_{0}}{{V}_{2}} + 1 )} \qquad ( 8 )$$

Using Equation (8), if the point (I₀, V₀) lies on AP1, then the fraction a=0, and if the point (I₀, V₀) lies on AP2, then the fraction a=1. If the point (I₀, V₀) lies between AP1 and AP2, the fraction a is between 0 and 1, being close to 0 to the extent that (I₀, V₀) is close to AP1.

In another implementation, the interpolation fraction a may be computed by measuring the fractional vertical distance between the point (I₀, V₀) and AP1. In such an implementation, the response intensity interval between the point where AP1 has stimulus intensity I₀ and the point where AP2 has stimulus intensity I₀ is partitioned into a sub-interval of fractional length a between AP1 and the point (I₀, V₀), and a sub-interval of fractional length 1-a between AP2 and the point (I₀, V₀). According to this implementation, the fraction a may be computed as follows:

$$a = \frac{{V}_{0} - {V}_{1} ( \frac{{I}_{0}}{{I}_{1}} - 1 )}{{V}_{2} ( \frac{{I}_{0}}{{I}_{2}} - 1 ) - {V}_{1} ( \frac{{I}_{0}}{{I}_{1}} - 1 )} \qquad ( 9 )$$

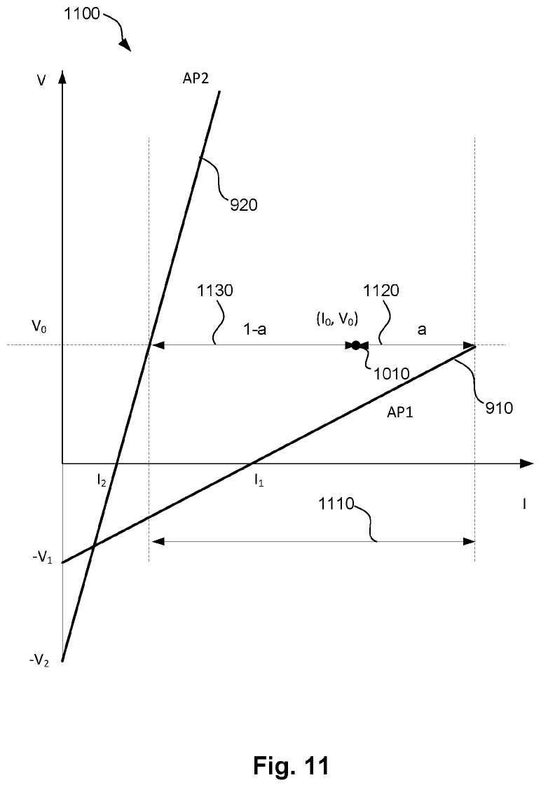

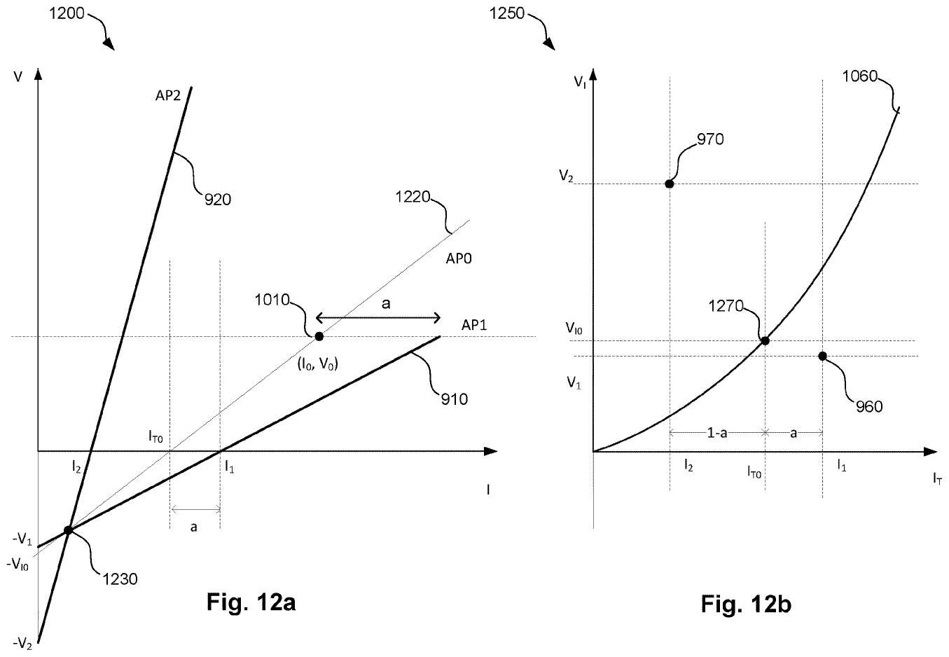

FIGS. 12a and 12b illustrate a method of interpolation that makes use of the fraction a according to one implementation. In FIG. 12b, the graph 1250 in IT-VI space shows the curve 1060 representing the point (I₀, V₀) 1010. The point (I_{T0}, V_{I0}) 1270 representing AP0 is the point on the curve 1060 that is a % of the horizontal distance from I1 to I2. That is, the threshold I₀ of AP0 may be computed as follows:

I_{T0}=I₁(1−a)+aI₂ (10)

The intercept V_{I0} may then be computed from the threshold I_{T0} using equation (7) as follows:

$${V}_{I 0} = \frac{{V}_{0} {I}_{T 0}}{{I}_{0} - {I}_{T 0}} \qquad ( 11 )$$

FIG. 12a is a graph in I-V space showing the resulting AP0 1220 with threshold I₀ and intercept V_{I0}. It may be seen from similar triangles that because I_{T0} is a % of the distance from I1 to I2, just as I₀ is a % of the horizontal distance from AP1 to AP2, therefore AP0 is the AP that passes through (I₀, V₀) and the intersection point 1230 of AP1 and AP2. This implementation (horizontal interpolation using the fractional horizontal distance) therefore implicitly assumes that all APs for the patient pass through a single intersection point 1230 and is therefore referred to as the single intersection point (SIP) solution.

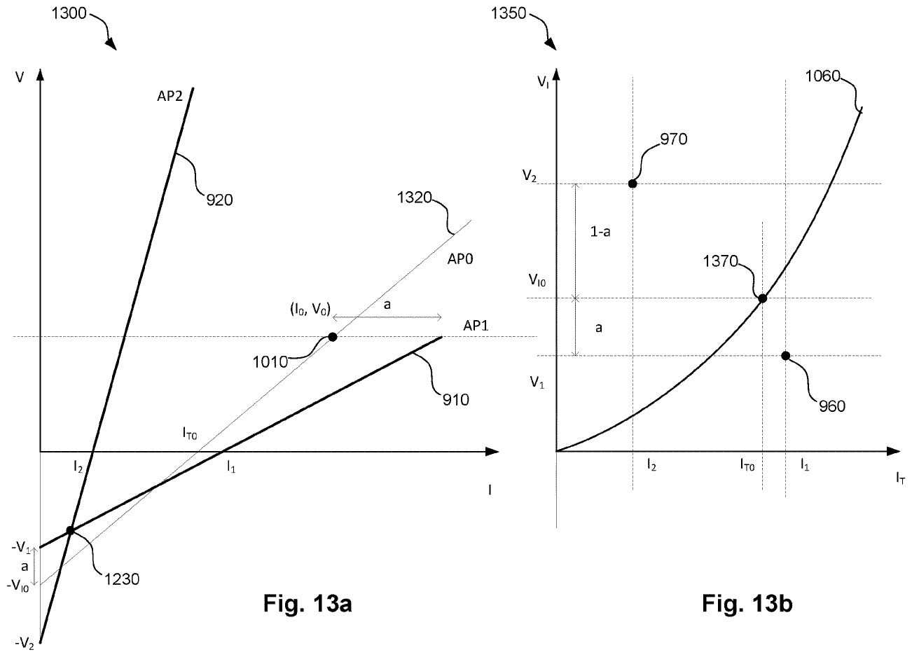

FIGS. 13a and 13b illustrate another method of interpolation that makes use of the fraction a according to another implementation. In FIG. 13b, the graph 1350 in IT-VI space shows the curve 1060 representing the point (I₀, V₀) 1010. The point (I_{T0}, V_{I0}) 1370 representing AP0 is the point on the curve 1060 that is a % of the vertical distance from V1 to V2. That is, the intercept V_{I0} of AP0 may be computed as follows:

V_{I0}=V₁(1−a)+aV₂ (12)

The threshold I_{T0} may then be computed from the intercept V_{I0} using equation (7) as follows:

$${I}_{T 0} = \frac{{I}_{0} {V}_{0}}{{V}_{0} + {V}_{I 0}} \qquad ( 13 )$$

FIG. 13a is a graph in I-V space showing the resulting AP0 1320 with threshold I₀ and intercept V_{I0}. As shown, V_{I0} is a % of the vertical distance from V1 to V2, whereas a is the fractional horizontal distance between (I₀, V₀) and AP1. It may be seen in FIG. 13a that AP0 1320 is different from the SIP solution (AP0 1220 in FIG. 12a) and therefore does not pass through the intersection point 1230 of AP1 and AP2. Likewise, if the fractional-vertical-distance implementation is used to compute the fraction a, and horizontal interpolation is used to interpolate, the resulting AP0 is also different from the SIP solution, as well as being different from the AP0 1320.

However, if the fractional-vertical-distance implementation is used to compute the fraction a, and vertical interpolation is used to interpolate, it may be shown from similar triangles that because V_{I0} is a % of the vertical distance from V_{I} to V2, just as V₀ is a % of the vertical distance from AP1 to AP2, therefore AP0 is the SIP solution (AP0 1220 in FIG. 12a, i.e. the AP that passes through (I₀, V₀) and the intersection point 1230 of AP1 and AP2).

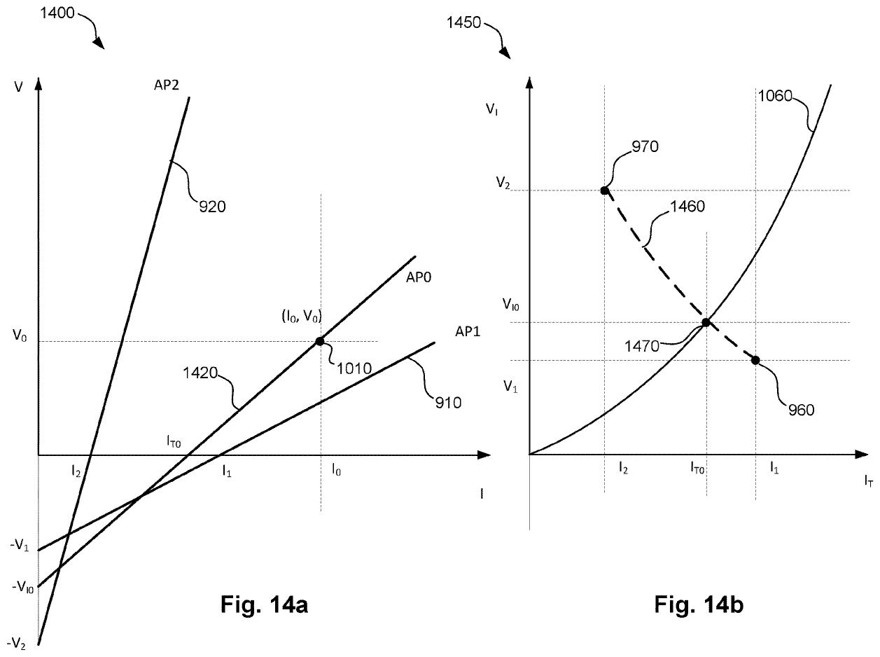

FIGS. 14a and 14b illustrate another method of interpolation according to another implementation that does not need a fraction a. In FIG. 14b, the graph 1450 in IT-VI space shows the curve 1060 representing the point (I₀, V₀) 1010. The point (I_{T0}, V_{I0}) 1470 representing AP0 is the point on the curve 1060 that lies on an interpolative path 1460 between the points 960 (I1, V1) and 970 (I2, V2) representing AP1 and AP2 respectively.

In one implementation, the interpolative path 1460 is a straight line between the points 960 (I1, V1) and 970 (I2, V2). The point (I_{T0}, V_{I0}) 1470 representing AP0 lies on this straight line provided the following equation is satisfied:

$${V}_{I 0} = {V}_{1} + ( {I}_{T 0} - {I}_{1} ) ( \frac{{V}_{2} - {V}_{1}}{{I}_{2} - {I}_{1}} ) \qquad ( 14 )$$

Finding the point (I_{T0}, V_{I0}) 1470 representing AP0 is therefore a matter of simultaneously solving Equation (11) and Equation (14). This results in a quadratic equation in I_{T0} that may be solved analytically.

More generally, the interpolative path 1460 may be written as a function ƒ in IT-VI space

V_{I}=ƒ(I_{T}) (15)
that pertains to the patient. An expression for the interpolative path 1460 as written in Equation (15) may be found by fitting the defining parameters of the reference APs for the patient to a curve in IT-VI space. Finding the point (I_{T0}, V_{I0}) 1470 representing AP0 is then a matter of simultaneously solving Equation (11) and Equation (15). Depending on the form of the interpolative path 1460, these simultaneous equations may be solved analytically or approximately, by iteration, according to conventional mathematical methods.

Another example of an interpolative path results from the above-mentioned assumption that all APs for the patient pass through a single intersection point 1230. The interpolated activation plot AP0 must also therefore pass through the single intersection point. Writing the coordinates of such a single intersection point 1230 as (I_{C}, −V_{C}), the interpolative path 1460 may be written using equation (7) as

$${V}_{I} = \frac{- {V}_{C} {I}_{T}}{{I}_{C} - {I}_{T}} \qquad ( 16 )$$

Finding the point (I₀, V_{I0}) 1470 representing AP0 is then a matter of simultaneously solving Equation (11) and Equation (16). The resulting threshold value, computable as

$${I}_{T 0} = \frac{{V}_{0} {I}_{C} + {V}_{C} {I}_{0}}{{V}_{C} + {V}_{0}} \qquad ( 17 )$$

is the SIP solution mentioned above.

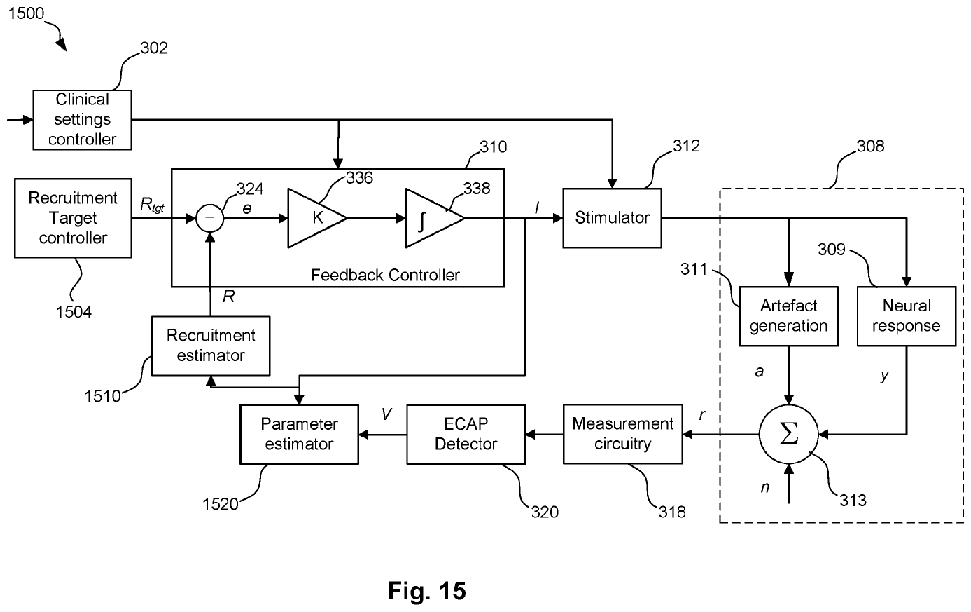

FIG. 15 is a schematic illustrating elements and inputs of a constant-recruitment CLNS system 1500, according to one implementation of the present technology. The constant-recruitment CLNS system 1500 is similar to the constant-ECAP CLNS system 300 of FIG. 5, with like labels indicating like elements. The main difference is that the feedback variable in the constant-recruitment CLNS system 1500 is the estimated recruitment R according to Equation (5) or (equivalently) Equation (6). The recruitment R is estimated by the recruitment estimator 1510, which estimates the recruitment R according to Equation (5) or Equation (6) from the defining parameters of the intermediate AP. The defining parameters are provided by the parameter estimator 1520, which takes as inputs the measured response intensity V from the ECAP detector 320 and the intensity I of the stimulus that evoked the response with intensity measured as V. The feedback controller 310 adjusts the stimulus intensity I to maintain the estimated recruitment R at or near a recruitment target R_{tgt}, in the same way that the feedback controller 310 in the constant-ECAP CLNS system 300 of FIG. 5 adjusts the stimulus intensity I to maintain the neural response intensity Vat or near a target response intensity V_{tgt}. The recruitment target R_{tgt} is provided by the recruitment target controller 1504 in the same way that the ECAP target controller 304 provides the target response intensity V_{tgt}.

The parameter estimator 1520 uses the reference APs constructed during programming in respective reference postures, and in particular AP1 and AP2 corresponding to the two extreme reference postures (least sensitive and most sensitive respectively), to estimate the defining parameters of the intermediate AP by interpolation between AP1 and AP2 in the manner described above. As described above, the defining parameters of each reference AP may be stored in the clinical settings 121 of the stimulator 100 by the CPA according to the present technology. In some implementations, the defining parameters of each reference AP may also be fit to an interpolative path ƒ in IT-VI space in the form of Equation (15). The interpolative path ƒ may be stored in the clinical settings 121 of the stimulator 100 by the CPA according to the present technology.

In one implementation, the defining parameter estimator 1520 first computes a fraction a as a fractional horizontal distance using Equation (8). The parameter estimator 1520 then computes the threshold I₀ or the intercept V_{I0} of the intermediate AP, AP0, from a by horizontal or vertical interpolation using Equation (10) or Equation (12). In such an implementation, the recruitment estimator 1510 then estimates recruitment R from the threshold I_{T0} using Equation (5) or the intercept V_{I0} using Equation (6).

In another implementation, the parameter estimator 1520 first computes a fraction a as the fractional vertical distance using Equation (9). The recruitment estimator 1510 then computes the threshold I₀ or the intercept V_{I0} of the intermediate AP, AP0, from a by horizontal or vertical interpolation using Equation (10) or Equation (12). In such an implementation, the recruitment estimator 1510 then estimates recruitment R from the threshold I_{T0} using Equation (5) or the intercept V_{I0} using Equation (6).

In another implementation, the parameter estimator 1520 simultaneously solves Equation (11) and Equation (14) (for a linear interpolative path) or Equation (11) and Equation (15) (for a general interpolative path ƒ) to obtain the point (I₀, V_{I0}) representing AP0. The recruitment estimator 1510 then estimates recruitment R from the threshold I_{T0} using Equation (5) or from the intercept V_{I0} using Equation (6).

It may be seen from inspection of Equations (1) and (2) that while a constant-recruitment CLNS system 1500 has been described above using threshold I_{T} and intercept V_{I} as the defining parameters of an AP, a constant-recruitment CLNS system may alternatively and equivalently be formulated in terms of threshold I_{T} and sensitivity S, or intercept V_{I} and sensitivity S, as the defining parameters of an AP, using the relation

$$S = \frac{{V}_{I}}{{I}_{T}} \qquad ( 18 )$$

among the three candidate parameters of an AP.

If the point (I₀, V₀) does not lie between the two reference APs, for example if it is measured during a sudden sharp postural disturbance such as a cough, the same methods as outlined above may be used, except that interpolation is substituted for extrapolation and the fraction a is no longer confined to the interval [0, 1].

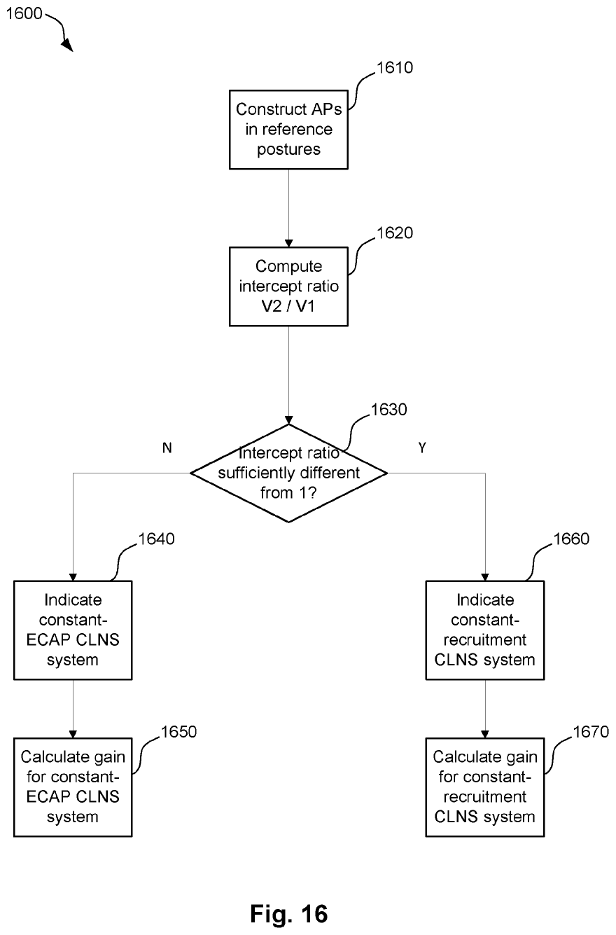

FIG. 16 is a flow chart illustrating a method 1600 of determining a suitable feedback controller for a patient. The method 1600 may be carried out by the CPA during programming of the patient. As mentioned above, the intercept ratio V2/V1 is the variation in recruitment R as defined in Equation (5) or Equation (6) that would be expected of a constant-ECAP CLNS system such as the CLNS system 300 that adjusted stimulus intensity I to maintain a constant response intensity V as posture changed from the least-sensitive (AP1) to the most-sensitive (AP2) posture, or vice versa. The method 1600 therefore uses the intercept ratio V2/V1 as a metric to determine whether a constant-ECAP CLNS system such as the CLNS system 300 is capable of maintaining constant or near-constant recruitment R as posture changes, or whether implementations of a constant-recruitment CLNS system such as the CLNS system 1500 are more suitable for maintaining constant or near-constant recruitment as posture changes.

The method 1600 starts at step 1610, which constructs reference APs in respective reference postures as described above. Step 1610 includes determining the intercept V_{I} for each reference AP, and in particular for AP1 and AP2 corresponding to the two extreme reference postures. These two intercepts are labelled V1 (for the least sensitive posture) and V2 (for the most sensitive posture).

Step 1620 then computes the intercept ratio V2/V1. Step 1630 then compares the intercept ratio V2/V1 to 1 to determine whether the intercept ratio V2/V1 is sufficiently different from 1 to indicate the superior suitability of a constant-recruitment CLNS system 1500 for the patient over a constant-ECAP CLNS system 300. In one implementation, step 1630 determines whether the absolute value of the difference between the intercept ratio V2/V1 and 1 is greater than a threshold of acceptable variation, for example 5%. If not (“N”), step 1640 indicates that a constant-ECAP CLNS system 300 is suitable for the patient.

Step 1650 then calculates a value for the gain K of the gain element 336 of the constant-ECAP CLNS system 300 from the maximum patient sensitivity S₂, that is, the slope of the reference AP corresponding to the most sensitive posture. In one implementation, step 1650 calculates the gain K as

$$K = \frac{cos ω - 1 + \sqrt{{cos}^{2} ω - 4 cos ω + 3}}{{S}_{2}} \qquad ( 19 )$$

where

$$ω = \frac{2 π {f}_{c}}{{f}_{s}} ,$$

ƒ_{c} is a loop cutoff frequency, and ƒₛ is the stimulus frequency. In one implementation, the loop cutoff frequency is set to 3 Hz. The indication from step 1640 and the gain K from step 1650 are stored by the CPA in the clinical settings data 121 as part of the therapy parameters for the patient.

If the difference between the intercept ratio V2/V1 and 1 is greater than the threshold of acceptable variation (“Y”), step 1660 indicates that a constant-recruitment CLNS system 1500 is suitable for the patient. Step 1670 then calculates a value for the gain K of the gain element 336 of the constant-recruitment CLNS system 1500 in similar fashion to step 1650, except using the maximum value R′ₘₐₓ of the derivative of recruitment R with respect to stimulus intensity I in place of the maximum patient sensitivity S₂. From Equation (5), the derivative R′ of recruitment R with respect to stimulus intensity I in any posture is the reciprocal of the threshold I_{T} in that posture. The maximum value R′ₘₐₓ of the derivative of recruitment R with respect to stimulus intensity I is therefore the reciprocal 1/I_{T2} of the threshold I_{T2} of the reference AP corresponding to the most sensitive posture. In one implementation, step 1670 calculates the gain K as

K=I_{T2}(cos ω−1+{square root over (cos²ω-4 cos ω+3))} (20)
where

$$ω = \frac{2 π {f}_{c}}{{f}_{s}} ,$$

ƒ_{c} is a loop cutoff frequency, and ƒₛ is the stimulus frequency. In one implementation, the loop cutoff frequency is set to 3 Hz.

The indication from step 1660 and the gain K from step 1670 are stored by the CPA in the clinical settings data 121 as part of the therapy parameters for the patient.

In an alternative implementation of the method 1600, step 1670 is omitted, so that a fixed gain K is not stored as part of the clinical settings 121. Instead, the constant-recruitment CLNS system 1500, e.g, via the clinical settings controller 302, computes the gain K for the gain element 336 “on the fly” using the current estimate of the threshold I_{T0} in Equation (20) at each adjustment of the stimulus intensity (i.e. at the stimulus frequency), or at some multiple of the stimulus frequency e,g, every 10 adjustments. The parameter estimator 1520 provides the estimated threshold I_{T0} to the clinical settings controller 302 for this purpose.

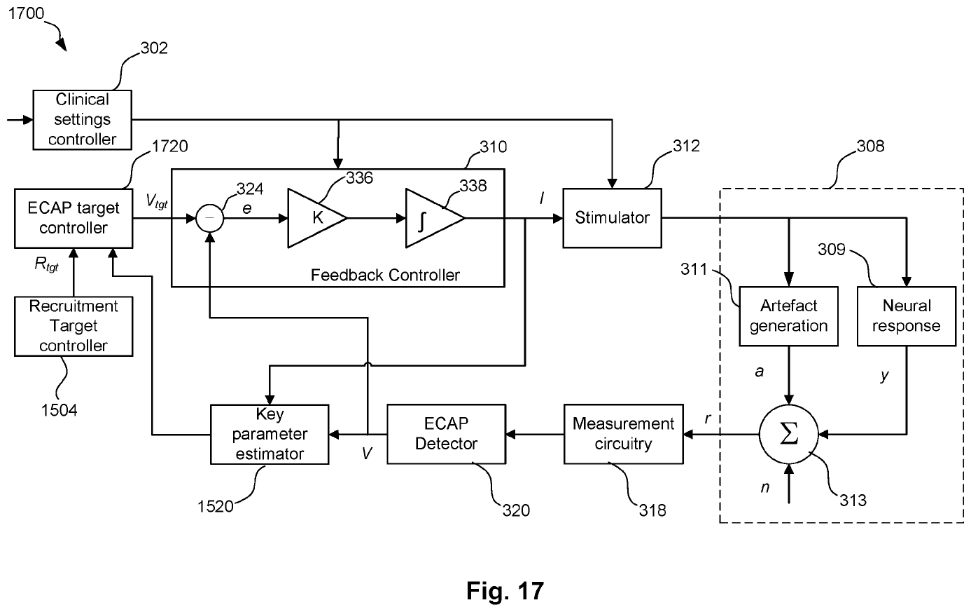

FIG. 17 is a schematic illustrating elements and inputs of a constant-recruitment CLNS system 1700, according to another implementation of the present technology. The constant-recruitment CLNS system 1700 is similar to the constant-recruitment CLNS system 1500 of FIG. 15, with like labels indicating like elements. The main difference is that the constant-recruitment CLNS system 1700 does not estimate recruitment R for use as a feedback variable, so the recruitment estimator 1510 is not present. Instead, the constant-recruitment CLNS system 1700 has an ECAP target controller 1720 that dynamically determines a target response intensity V_{tgt} based on the defining parameters of the intermediate AP provided by the parameter estimator 1520, and the recruitment target R_{tgt} provided by the recruitment target controller 1504. The ECAP target controller 1720 dynamically determines the target response intensity V_{tgt} based on equation (6) as follows:

V_{tgt}=R_{tgt}V_{I0} (21)

The feedback controller 310 of the constant-recruitment CLNS system 1700 adjusts the stimulus intensity I to maintain the measured response intensity V to be at or near the dynamically determined target response intensity V_{tgt} as described above. In this way the constant-recruitment CLNS system 1700 maintains the recruitment (without ever estimating it explicitly) to be at or near the recruitment target R_{tgt}.

It will be appreciated by persons skilled in the art that numerous variations or modifications may be made to the invention as shown in the specific implementations without departing from the spirit or scope of the invention as broadly described. The present implementations are, therefore, to be considered in all respects as illustrative and not limiting or restrictive.

### INDUSTRIAL APPLICABILITY

It is apparent from the above that the arrangements described are applicable to the health care industries.

LABEL LIST

| | |
|---|---|
| stimulator | 100 |
| patient | 108 |
| electronics module | 110 |
| battery | 112 |
| telemetry module | 114 |
| controller | 116 |
| memory | 118 |
| clinical data | 120 |
| clinical settings | 121 |
| control programs | 122 |
| pulse generator | 124 |
| electrode selection module | 126 |
| measurement circuitry | 128 |
| ground | 130 |
| electrode array | 150 |
| biphasic stimulus pulse | 160 |
| ECAP | 170 |
| nerve | 180 |
| communications channel | 190 |
| external device | 192 |
| CLNS system | 300 |
| clinical settings controller | 302 |
| ECAP target controller | 304 |
| box | 308 |
| box | 309 |
| controller | 310 |
| box | 311 |
| stimulator | 312 |
| element | 313 |
| measurement circuitry | 318 |
| signal window | 319 |
| ECAP detector | 320 |
| comparator | 324 |
| gain element | 336 |
| integrator | 338 |
| activation plot | 402 |
| recruitment threshold | 404 |
| discomfort threshold | 408 |
| therapeutic range | 412 |
| point | 414 |
| activation plot | 502 |
| activation plot | 504 |
| activation plot | 506 |
| recruitment threshold | 508 |
| recruitment threshold | 510 |
| recruitment threshold | 512 |
| target ECAP amplitude | 520 |
| ECAP | 600 |
| graph | 700 |
| activation plot | 702 |
| activation plot | 704 |
| activation plot | 706 |
| dashed line | 708 |
| dashed line | 710 |
| dashed line | 712 |
| dashed line | 714 |
| recruitment threshold | 716 |
| recruitment threshold | 718 |
| recruitment threshold | 720 |
| graph | 800 |
| point | 810 |
| curve | 820 |
| graph | 900 |
| reference activation plot | 910 |
| reference activation plot | 920 |
| graph | 950 |
| point | 960 |
| point | 970 |
| graph | 1000 |
| graph | 1050 |
| curve | 1060 |
| graph | 1100 |
| interval | 1110 |
| sub - interval | 1120 |
| sub - interval | 1130 |
| intersection point | 1230 |
| graph | 1250 |
| graph | 1350 |
| graph | 1450 |
| interpolative path | 1460 |
| CLNS system | 1500 |
| target controller | 1504 |
| recruitment estimator | 1510 |
| parameter estimator | 1520 |
| method | 1600 |
| step | 1610 |
| step | 1620 |
| step | 1630 |
| step | 1640 |
| step | 1650 |
| step | 1660 |
| step | 1670 |
| CLNS system | 1700 |
| ECAP target controller | 1720 |

1. An implantable neuromodulation device for controllably delivering neural stimuli, the device comprising:
a stimulus source configured to provide neural stimuli to be delivered via one or more stimulus electrodes of an electrode array to a neural pathway of a patient in order to evoke a neural response from the neural pathway;
measurement circuitry configured to capture signal windows from signals sensed on the neural pathway via one or more measurement electrodes of the electrode array subsequent to respective neural stimuli; and
a control unit configured to:
control the stimulus source to provide a neural stimulus according to a stimulus intensity parameter;
measure an intensity of an evoked neural response in a captured signal window subsequent to the neural stimulus;
estimate one or more defining parameters of an intermediate activation plot corresponding to a current posture of the patient from the measured intensity of the evoked neural response, the stimulus intensity parameter, and a plurality of reference defining parameters of activation plots corresponding to respective reference postures; and
adjust, using the one or more estimated defining parameters, the stimulus intensity parameter so as to maintain a recruitment value of the provided neural stimuli at or near a recruitment target value.

2. The implantable neuromodulation device of claim 1, wherein the control unit is configured to adjust the stimulus intensity parameter by:
estimating a recruitment value of the provided neural stimulus from:
one of the one or more estimated defining parameters, and
one of the stimulus intensity parameter and the measured intensity; and
adjusting, using the estimated recruitment value, the stimulus intensity parameter so as to maintain the estimated recruitment value at or near the recruitment target value.

3. The implantable neuromodulation device of claim 2, wherein the one of the one or more estimated defining parameters of the intermediate activation plot comprises a recruitment threshold.

4. The implantable neuromodulation device of claim 2, wherein the one of the one or more estimated defining parameters of the intermediate activation plot comprises a value of response intensity at which the intermediate activation plot intercepts a response intensity axis.

5. The implantable neuromodulation device of claim 1, wherein the control unit is configured to adjust the stimulus intensity by:
determining a target response intensity from one of the one or more estimated defining parameters and the recruitment target value; and
adjusting, using the measured intensity, the stimulus intensity parameter so as to maintain the measured intensity at or near the determined target response intensity.

6. The implantable neuromodulation device of claim 5, wherein the one of the one or more estimated defining parameters of the intermediate activation plot comprises a response intercept, wherein the response intercept is the negative of the value of response intensity at which the intermediate activation plot intercepts a response intensity axis.

7. The implantable neuromodulation device of claim 1, wherein the control unit is configured to estimate the one or more defining parameters by interpolating between the reference defining parameters of a first reference activation plot of the plurality of reference activation plots and the reference defining parameters of a second reference activation plot of the plurality of reference activation plots.

8. The implantable neuromodulation device of claim 7, wherein the interpolating comprises interpolating a recruitment threshold of the intermediate activation plot between a recruitment threshold of the first reference activation plot and a recruitment threshold of the second reference activation plot.

9. The implantable neuromodulation device of claim 7, wherein the interpolating comprises interpolating a response intercept of the intermediate activation plot between the response intercept of the first reference activation plot and the response intercept of the second reference activation plot.

10. The implantable neuromodulation device of claim 8, wherein the interpolating uses an interpolation fraction representing the fractional distance between the stimulus intensity parameter and a stimulus intensity parameter value of the first reference activation plot.

11. The implantable neuromodulation device of claim 7, wherein the interpolating comprises jointly interpolating a recruitment threshold and a response intercept of the intermediate activation plot using an interpolative path between the first reference activation plot and the second reference activation plot.

12. An automated method of controllably delivering neural stimuli to a neural pathway of a patient, the method comprising:
delivering a neural stimulus to the neural pathway of the patient in order to evoke a neural response from the neural pathway, the neural stimulus being delivered according to a stimulus intensity parameter;
capturing a signal window sensed on the neural pathway subsequent to the delivered neural stimulus;
measuring an intensity of a neural response evoked by the delivered neural stimulus in the captured signal window;
estimating one or more defining parameters of an intermediate activation plot corresponding to a current posture of the patient from the measured intensity of the evoked neural response, the stimulus intensity parameter, and a plurality of reference defining parameters of activation plots corresponding to respective reference postures; and
adjusting, using the one or more estimated defining parameters, the stimulus intensity parameter so as to maintain a recruitment value of the delivered neural stimuli at or near a recruitment target value.

13. The method of claim 12, wherein the adjusting comprises:
estimating a recruitment value of the neural stimulus from the measured intensity, the stimulus intensity parameter, and one of the one or more defining parameters; and
adjusting, using the estimated recruitment value, the stimulus intensity parameter so as to maintain the estimated recruitment value at or near the recruitment target value.

14. The method of claim 13, wherein the one of the one or more estimated defining parameters comprises a recruitment threshold.

15. The method of claim 13, wherein the one of the one or more estimated defining parameters comprises a value of response intensity at which the intermediate activation plot intercepts a response intensity axis.

16. The method of claim 12, wherein the adjusting comprises:
determining a target response intensity from one of the one or more estimated defining parameters and the recruitment target value; and
adjusting, using the measured intensity, the stimulus intensity parameter so as to maintain the measured intensity at or near the determined target response intensity.

17. The method of claim 16, wherein the one of the one or more estimated defining parameters comprises a response intercept, wherein the response intercept is the negative of the value of response intensity at which the intermediate activation plot intercepts a response intensity axis.

18. The method of claim 12, wherein the estimating the one or more defining parameters comprises interpolating between the reference defining parameters of a first reference activation plot of the plurality of reference activation plots and the reference defining parameters of a second reference activation plot of the plurality of reference activation plots.

19. The method of claim 18, wherein the interpolating comprises interpolating a recruitment threshold of the intermediate activation plot between a recruitment threshold of the first reference activation plot and a recruitment threshold of the second reference activation plot.

20. The method of claim 18, wherein the interpolating comprises interpolating a response intercept of the intermediate activation plot between the response intercept of the first reference activation plot and the response intercept of the second reference activation plot.

21. The method of claim 19, wherein the interpolating uses an interpolation fraction representing the fractional distance between the stimulus intensity parameter and a stimulus intensity parameter value of the first reference activation plot.

22. The method of claim 18, wherein the interpolating comprises jointly interpolating a recruitment threshold and a response intercept of the intermediate activation plot using an interpolative path between the first reference activation plot and the second reference activation plot.

23. (canceled)

24. A closed-loop neural stimulation device for controllably delivering neural stimuli, the device comprising:
a control unit configured to repeatedly adjust a stimulus intensity parameter of provided neural stimuli so as to maintain a recruitment value of the provided neural stimuli at or near a recruitment target value,
wherein the control unit is configured to adjust the stimulus intensity parameter by:
estimating one or more defining parameters of an intermediate activation plot corresponding to a current posture from:
a measured intensity of an evoked neural response to a delivered neural stimulus,
a stimulus intensity parameter of the delivered neural stimulus, and
a plurality of reference defining parameters of activation plots corresponding to respective reference postures; and
adjusting, using the one or more estimated defining parameters, the stimulus intensity parameter so as to move the recruitment value closer to the recruitment target value.

25-40. (canceled)
